(19) United States
(12) Patent Application Publication
(10) Pub. No.: US 2018/0147077 A1
(43) Pub. Date: May 31, 2018

(54) BALLOON CATHETER HAVING AN INSERTION AID FOR A GUIDE WIRE

(71) Applicant: B. BRAUN MELSUNGEN AG, Melsungen (DE)

(72) Inventors: Karl-Heinz Scharschmidt, Berlin (DE); Jorge Calisse, Berlin (DE); Henning Wöbken, Mahlow (DE); Peter Günther, Berlin (DE)

(21) Appl. No.: 15/316,384

(22) PCT Filed: Jun. 3, 2015
(86) PCT No.: PCT/EP2015/062415

§ 371 Date: Dec. 5, 2016

(30) Foreign Application Priority Data
Jun. 6, 2014 (EP) 14171435.2

(51) Int. Cl. A61F 2/958; A61M 25/10; A61M 25/06; A61M 25/01

(52) U.S. Cl. CPC A61F 2/958; A61M 25/104; A61M 2025/0177; A61M 25/0668; A61M 2025/1081; A61M 2025/0681

(57) ABSTRACT

A protective sleeve for a balloon catheter includes an insertion aid for a guide wire. The insertion aid is integrated in the protective sleeve at a distal end of the protective sleeve. In addition, a balloon catheter includes a protective sleeve which at least partially surrounds a balloon. An insertion aid for a guide wire is integrated in the distal end of the protective sleeve.

The invention relates primarily to a balloon catheter, in particular a balloon catheter with stent, wherein the catheter has a protective sleeve which at least partially surrounds the balloon and optionally also the stent. Moreover, the invention also relates to a protective sleeve for a balloon catheter, and to a method for inserting a guide wire into the interior of a balloon catheter.

Catheters are used in medicine for various diagnostic or therapeutic purposes. They are generally inserted into hollow organs such as the stomach and intestines, but also into blood vessels or into the heart via the blood stream, in order in this way to permit diagnosis or therapy in situ.

Catheters referred to as balloon catheters are used in particular in percutaneous transluminal angioplasty (PTA) and in percutaneous transluminal coronary angioplasty (PTCA). These terms include techniques for widening a narrowed blood vessel or coronary vessel or for re-opening an occluded (generally peripheral) blood vessel or coronary vessel.

The corresponding techniques are described in more detail below, taking PTCA as a non-limiting example.

Usually, in PTCA, a catheter known as a guide catheter, generally with access through the inguinal artery, is guided against the blood stream toward the heart and is there introduced into the opening of the coronary artery. The balloon catheter is then advanced through the interior of the guide catheter. The balloon, with which the constriction in the coronary vessel in question is expanded, is located at the distal end of the balloon catheter. Following the expansion with the balloon, a vascular support is often inserted, which is intended to reduce the risk of renewed vascular occlusion. These vascular supports are thin wire meshes (referred to as stents), which are fitted on a balloon catheter in the contracted state. By the expansion of the balloon, the stent is pressed onto the wall of the vessel, and this vessel is thereby kept open.

To be able to perform the PTCA, a thin, flexible wire, referred to as a guide wire, has to be engaged past the vascular constriction (with X-ray monitoring).

This guide wire serves as a kind of guide rail, along which the balloon catheter is later moved into the constriction.

Such guide wires are very thin and generally have diameters of far below 1 mm.

In order to proceed as outlined above, it is necessary, prior to the insertion of the balloon catheter into the body, for the corresponding guide wire to be inserted (distally) into the balloon catheter itself. Only then can the guide wire perform its function as a guide rail for the balloon catheter.

This insertion of the guide wire into the balloon catheter proves difficult, since the guide wire having the stated small diameters has to be inserted into the catheter interior which has an only slightly greater diameter. For example, in such cases in cardiology, it is necessary to insert (thread) a guide wire with a diameter of 0.36 mm into a tube/catheter with an internal diameter of ca. 0.4 mm. This is complicated, particularly under poor lighting, and demands good visual acuity and a steady hand.

A further problem is that although balloon catheters generally have a protective sleeve that protects the balloon itself, and an optionally present stent, from damage, customary protective sleeves necessarily have to be removed prior to the insertion of the guide wire, with the result that, during the insertion of the guide wire, the balloon and optionally the stent may become damaged by the latter or by some other agency due to the required handling. This is particularly important in the case of balloons and stents that are additionally coated. These coatings can be, for example, so-called functional coatings, which make the coated surfaces suitable for specific applications, for example a hydrophilic coating, or coatings composed of or having active substances, in particular medicaments. In such cases, the coating may be at least partially damaged or come away.

In this connection, it will be briefly explained how the user of a balloon catheter normally proceeds for inserting a guide wire into the corresponding catheter.

If no insertion aid is present, the user takes the balloon catheter in one hand in such a way that the guide end, i.e. the front end of the catheter or the tip of the catheter, lies on the inner aspect of the pad of a finger, in particular of the index finger.

Then, with the other hand, the (rear) end of the guide wire is likewise laid on the corresponding inner aspect of the finger pad, as far as possible in a precise axial continuation of the catheter.

The user then pushes said end of the guide wire onto the balloon catheter and tries to push/thread the guide wire into the opening of the balloon catheter.

As has already been explained, this approach has the disadvantage, for example, that laying an optionally coated balloon catheter, in particular a balloon catheter coated with medicaments, onto the finger pad invariably causes this coating to partially rub off in the area of the finger. Consequently, the dose of medicament on the balloon catheter is reduced in an undesirable and unpredictable manner.

Moreover, the described pushing/threading approach requires a steady hand and a good eye, particularly since the opening that has to be aimed at during the insertion is relatively small.

So-called insertion aids for guide wires of catheters are known from the prior art. These are separate devices that have to be mounted distally on the catheter and, by virtue of their design, are intended to facilitate the insertion of the guide wire into the catheter. By way of example, reference is made here to the following patent publications: U.S. Pat. No. 5,978,699, WO-A2-2008/036842 and WO-A1-2012/115753.

However, following their use, the described insertion aids themselves are likewise located on the guide wire between the catheter and the patient, and it is therefore necessary to be able to remove the insertion aids again without having to pull the guide wire out of the catheter (or out of the patient).

Moreover, separate insertion aids of this kind also fail to solve the problem of possible damage to the balloon and optionally the stent. The use of these insertion aids also requires that an optionally present protective sleeve be removed prior to the insertion of the guide wire. The risk of damage to the balloon or stent, in particular the risk of damage to coatings on the balloon or stent, is therefore also present here.

Accordingly, the object of the invention is to minimize the risk of damage to the balloon or stent of a balloon catheter by comparison with previously known designs. At the same time, the insertion of the guide wire into the catheter should itself be as simple as possible. The physician, particularly when performing PTA or PTCA, should not have to make unnecessary or difficult maneuvers.

This object is achieved by the balloon catheter with the features of claim 1 and by the protective sleeve with the features of claim 15. The invention also comprises the method described in claim 17 for inserting a guide wire into a balloon catheter. Preferred embodiments of this balloon catheter, of this protective sleeve and of this method are defined in the dependent product claims.

The wording of all the claims is herewith incorporated by reference into the content of this description.

According to the invention, a “balloon catheter” is understood as a catheter produced mostly from plastic and having a deployable balloon, which is generally located at the tip of the catheter. If appropriate, the balloon can also have a (functional) coating, preferably with at least one medicament or at least one other active substance. A balloon catheter often has at least two and sometimes several cavities (lumens), which extend in the longitudinal direction of the balloon catheter. An inner lumen receives the guide wire, and a lumen arranged farther to the outside has the inflating medium for the balloon. Balloon catheters are used especially in angioplasty, i.e. in the widening of narrowed vessels.

The term “stent” designates a medical implant in the form of a vascular support which is introduced into hollow organs in order to keep them open. Such stents are in most cases constructed as lattice-like tubes, which can be made of metal and/or plastic. Moreover, such stents can be functionally coated, in particular with medicaments or other active substances.

According to the invention, the term “protective sleeve” is intended to cover all items in the form of a sleeve or sheath that at least partially surround the balloon catheter (directly) and thus shield or protect the corresponding parts of the balloon catheter from external influences. Preferably, the balloon catheter, and optionally also an additionally provided stent, is surrounded substantially completely, in particular completely, i.e. in its entirety, by the protective sleeve.

According to the invention, a “guide wire” designates a wire (or optionally also an item with a similar function, for example a spring tube or the like) which, in the manner of a guide rail, can serve for the movement of a balloon catheter inside a hollow organ, in particular inside a vessel.

The terms “distal” and “proximal” used in this application are to be understood in the sense of a designation of position and direction in relation to the user of the catheter, i.e. for example the physician. Here, “distal” means directed away from, and “proximal” means close to. Accordingly, when reference is made to a distal end of the protective sleeve or of the catheter, this means that, in relation to the functional state of the corresponding item, the end in question forms the front end of the protective sleeve or of the catheter, which end is “directed away from the user (physician)”. The user (physician) then takes hold of the catheter for example at the proximal (rear) end of the protective sleeve or of the catheter.

According to claim 1 of the present invention, in a balloon catheter which preferably additionally has a stent, a protective sleeve is provided which at least partially surrounds, preferably substantially completely surrounds, the balloon and optionally also the stent. According to the invention, an insertion aid, i.e. a device facilitating the insertion of a guide wire into the balloon catheter, i.e. into the corresponding lumen of the balloon catheter, is integrated in the distal end of the protective sleeve.

This means that, in contrast to the prior art, no separate insertion aid is needed, and instead such an insertion aid is already present at the distal end of the protective sleeve. Accordingly, embodiments according to the invention are also possible in which the insertion aid is already pre-fitted on the protective sleeve or is formed in one piece with this protective sleeve.

To be able then to insert the guide wire into the interior of the catheter with in fact the help of the insertion aid, a corresponding opening has to be created on the insertion aid integrated in the protective sleeve or this opening can preferably already be provided on the insertion aid. In the latter case, no further and possibly difficult measures are then required to create such an opening on the insertion aid in the first place. In this connection, it is also possible that means allowing the opening to be easily formed are provided on the insertion aid. For example, predetermined breaking points or perforations or incisions can be present in the protective sleeve, by means of which a guide wire can easily penetrate the protective sleeve at the corresponding site and can thus automatically create the opening required for the insertion.

In connection with the observations last made, it is also optionally possible to provide, on the protective sleeve, an item or a tool which either keeps a corresponding opening on the insertion aid open or makes it easier to create such an opening. Such a means can also serve (optionally in addition to the already described function in relation to the opening on the insertion aid) to prevent accidental removal of the protective sleeve from the catheter.

Said means can, for example, be a tool in the manner of a stylet which, on the one hand, can open or can keep open the opening on the protective sleeve for the guide wire and, on the other hand, prevents accidental removal of the protective sleeve from the catheter, for example through slipping out of place.

In one development, the insertion aid integrated in the protective sleeve is preferably duct-like or tubular. In this way, the elongate guide wire can be easily guided inside the insertion aid in the direction of the interior of the catheter. It is further preferable if the insertion aid widens like a funnel in the direction of the opening provided at the distal end, i.e. has a greater cross-sectional surface area in the direction of this opening than in the direction of the interior of the catheter. In this way, the guide wire can be more easily inserted (threaded) into the duct-like or tubular part of the insertion aid.

The shape of the cross-sectional surface area of a duct-like or tubular insertion aid (including the funnel-like widenings) is not critical according to the invention, and it is therefore possible, for example, to have cross-sectional surface areas that are triangular, rectangular, square or oval or of any other desired shape. Preferably, the corresponding cross-sectional surface areas are circular, such that they correspond to the generally circular cross-sectional surface area of guide wires.

According to the invention, embodiments of the balloon catheter are preferred in which a recess is configured in the manner of a trough or of an open duct at the distal end of the insertion aid. If appropriate, this recess can also be formed integrally at this end of the insertion aid.

This trough-like or duct-like recess serves to (further) facilitate the insertion of the guide wire, i.e. the described pushing or threading into the corresponding opening, here into the opening of the insertion aid. The recess open at one end, i.e. the recess generally open at the top and preferably also at one side in the state of use, has in fact the advantage that the guide wire can be fitted into this recess from the side and/or from the top. In this way, the guide wire is correctly positioned, as it were automatically, with respect to the opening into which it is to be pushed/threaded. Bringing the guide wire axially to the corresponding opening, with precise targeting of the corresponding opening, is avoided in this way.

In the preferred embodiments described, the described recess can in principle have any desired cross-sectional surface area. In particular, preference is given to a cross-sectional surface area in the shape of a segment of a circle, preferably a cross-sectional surface area in the shape of a semicircle, or a V-shaped cross-sectional surface area. With cross-sectional surface areas such as these, the guide wire, after introduction/insertion into the recess, will settle in the lower area of the recess and, with the guiding thereby achieved, is thus movable in the direction of the opening.

With such a recess, in particular a recess with said preferred cross-sectional surface areas, the “capturing surface” for the guide wire can be considerably increased, with the same amount of material, compared to cone-shaped funnels, since such a recess can as a whole be made shallower. Moreover, a geometric offset of the recess in the axial direction (in relation to the geometry of the rest of the insertion aid) could also be accepted, since such an offset does not have a negative impact on the pushing/threading process.

It is moreover advantageous according to the invention, in said preferred embodiments, if the cross-sectional surface area of the recess increases, in particular continuously increases, toward the distal end of the insertion aid. Particularly if the guide wire is placed in the recess from the side, the penetration of the guide wire into the insertion aid is further facilitated thereby.

As has already been described, the recess provided according to the invention makes it easier to push/thread the guide wire into the opening of the insertion aid. Accordingly, it is further preferred if this opening is flush with the lowest point of the cross-sectional surface area of the recess, such that the guide wire introduced into the recess necessarily slides into this opening, which can be assisted by a suitable configuration of the corresponding end of the recess toward the opening. Accordingly, the opening of the balloon catheter into which the guide wire is then guided from the insertion aid then also lies preferably level with the lowest point of the cross-sectional surface area of the recess.

In the preferred embodiments of the balloon catheter according to the invention with a recess at the distal end of the insertion aid, it is further preferred if this recess is coded, in particular coded so as to improve its visual discernibility to the user. Such a coding is in particular a color coding. This (color) coding makes it easier for the user to identify the recess into which the guide wire is to be placed or inserted.

This coding, in particular this color coding, is preferably provided on the inner face of the recess, i.e. on the face of the recess with which the guide wire comes into contact.

In a development of the balloon catheter according to the invention, it is preferable if the protective sleeve has, proximally adjacent to the insertion aid, at least one area that is flattened in relation to the shape of the rest of the protective sleeve. In the state of use of the insertion aid, this flattened area can be located at least on the downwardly directed face of the protective sleeve, i.e. on the side which comes into contact, for example in the described manner, with the finger of the user. Preferably, the corresponding flattening can be located not only on this lower face of the insertion aid, but also on the top face thereof, particularly in view of such a flattened shape being easier to produce.

By virtue of said flattening, the corresponding area of the insertion aid/protective sleeve can be easily positioned on the pad of a user's finger. In this way too, the balloon catheter does not easily slip away from the finger pad. The handling of the balloon catheter according to the invention is thus improved overall.

The insertion aid can preferably widen in a funnel shape also in the proximal direction, i.e. in the direction of its proximal end. This also facilitates the receiving and securing of the catheter, which of course take place at this proximal end of the insertion aid. Here too, the shape of a cross-sectional surface area of the funnel-like widening is preferably coordinated with the shape of a cross-sectional surface area of the catheter, these cross-sectional surface areas in this case also being circular cross-sectional surface areas in particular.

In the balloon catheters claimed according to the invention, it is expedient if the removal of the protective sleeve takes place (only) after insertion of the guide wire into the interior of the catheter. According to the invention, it is thus advantageous if, in preferred embodiments of the balloon catheter according to the invention, means for removal of the protective sleeve from the catheter are provided on said protective sleeve. Such means are preferably provided in the longitudinal direction of the protective sleeve (with respect to the longitudinal direction of the catheter and of the guide wire inserted therein). Such means arranged in the longitudinal direction facilitate the removal of the protective sleeve, since in this way the protective sleeve can be easily drawn or peeled away from the guide wire and/or from the catheter. This is illustrated more clearly in connection with the drawing.

The means for removing the protective sleeve from the catheter (and/or from the optionally already inserted guide wire) are preferably in the form of at least one predetermined breaking point in the protective sleeve, or at least one perforation in the protective sleeve, or at least one incision in the protective sleeve. Advantageously, several of said means can also be provided simultaneously on the protective sleeve or in the protective sleeve.

All of said means have in common that, with them, the protective sleeve can be easily opened around the catheter and optionally around the stent and/or around the guide wire and can be easily detached from these. In this way, the protective sleeve can be made from a stable, mechanically strong material, which can reliably protect the balloon and its coating from damage. By using a strong (rigid) material of this kind, it is also possible to avoid or at least reduce an in principle undesirable contact between the protective sleeve and the outer surface of the balloon. A (subsequent) opening, cutting open or such like of the protective sleeve, for which tools such as knives, scissors and the like are needed, is not necessary.

In a development of the embodiments of the balloon catheter according to the invention in which means for removal of the protective sleeve are provided, a kind of handle is additionally provided for these means. With the aid of this handle, which can preferably be designed in the manner of a tab or a notch, the means for removal of the protective sleeve can be more easily actuated. For example, a user can take hold of the balloon catheter by the corresponding tab or notch and, in this way, the protective sleeve can then be easily separated or torn open, for example by way of perforations present there.

In all the embodiments of the invention, said protective sleeve can be made of various materials, with plastics preferably being used. Materials such as polytetrafluoroethylene, polyethylene, polypropylene or polyether block amide are mentioned here purely by way of example.

To prevent the user from inserting the balloon catheter into the body of the patient without detaching the protective sleeve from the catheter, the protective sleeve can be visually coded or colored, for example with conspicuous (signal) colors and/or patterns. A deliberately enlarged diameter of the protective sleeve along at least part of its length can also fulfill this (safety) function.

Finally, the balloon catheter according to the invention can preferably be located inside a sterile package. Accordingly, a sterile package of this kind, in which a balloon catheter according to the invention is contained, also forms subject matter of the invention.

Moreover, the invention also comprises the protective sleeve with integrated insertion aid, which has already been described in connection with the balloon catheter according to the invention. Such a protective sleeve is provided for a balloon catheter, in particular for a balloon catheter with stent, and it at least partially surrounds, preferably substantially completely surrounds, the balloon and optionally also the stent. According to the invention, an insertion aid for a guide wire is integrated in the protective sleeve, in particular in the distal end of the protective sleeve.

In one development, the protective sleeve can advantageously be designed as has already been described in connection with the preferred embodiments of the balloon catheter according to the invention. This relates therefore to the features of the opening in the insertion aid for insertion of the guide wire, the duct-like or tubular design of the insertion aid, optionally with a funnel-like widening in the distal and/or proximal direction. In this connection, reference is also made in particular to all the embodiments of the insertion aid having a trough-like or duct-like recess. The described means for removal of the protective sleeve from the catheter, and the handle disclosed in this connection, are also hereby intended by reference to be part of the description in connection with the features of the protective sleeve.

Finally, the invention also comprises a method for inserting a guide wire into the interior of a balloon catheter, in particular of a balloon catheter with stent. In said method, a balloon catheter, or a balloon catheter with stent, is made available, this balloon catheter having a protective sleeve which at least partially surrounds, preferably substantially completely surrounds, the balloon and optionally also the stent. An insertion aid (according to the invention) is additionally integrated in the distal end of the protective sleeve. According to the method, the guide wire is then inserted distally into the protective sleeve via the insertion aid.

The described invention is associated with a whole series of advantages.

Thus, a protective sleeve according to the invention with an integrated insertion aid can be used for very different balloon catheters. These can be balloon catheters with or without stent. The use of the protective sleeve according to the invention is particularly preferable in connection with coated balloon catheters, in particular balloon catheters coated with medicaments, wherein either the balloon or (alternatively or additionally) an optionally present stent can be coated.

An important advantage of the invention is that, compared to separate insertion aids as are known from the prior art, in the case of the invention the balloon of the balloon catheter, or also a stent if present, does not have to be touched during the insertion of the guide wire. With correct handling of the balloon catheter according to the invention and of the protective sleeve according to the invention, it is indeed impossible to touch the balloon or the stent during the insertion/threading of the guide wire. The protective sleeve with integrated insertion aid, particularly in preferred embodiments, completely covers all the other component parts of the balloon catheter according to the invention. Said advantage is seen particularly in coated/medicament-coated balloon catheters (with or without stent). Here, touching should be completely avoided during handling, i.e. in the present case in particular during the insertion of the guide wire. Otherwise, the applied layers, in particular the applied medicament layers, would be damaged and, consequently, the function of the coating would be at least impaired, if not prevented.

A further advantage that should be mentioned is that, for the person using the balloon catheter according to the invention, the handling during the insertion of the guide wire is made considerably easier. No separate insertion aid first has to be attached in order to actually permit the insertion. The integrated insertion aid, particularly in the embodiments with a trough-like or duct-like recess, means that it is possible to begin immediately with the insertion of the guide wire.

In the latter connection, it should also be noted that the integration of the insertion aid in the protective sleeve is generally easy to do during the production of the protective sleeve. Accordingly, compared to the use of a separate insertion aid, the costs met by the user are generally reduced.

The advantages mentioned, and further advantages, will become clear from the following description in conjunction with the drawings and with the example. The features described there can be implemented in the invention either individually or in combination.

In the drawing:

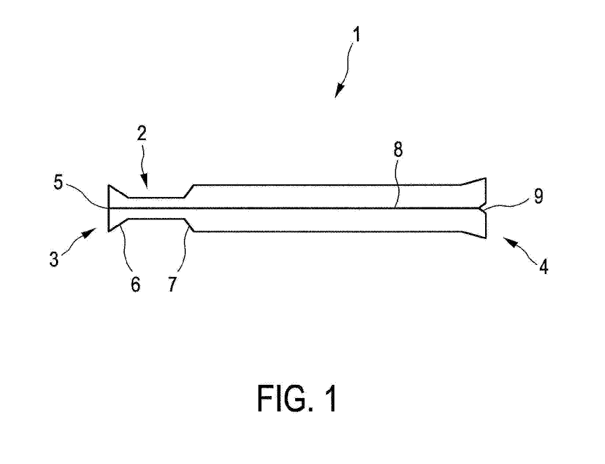

FIG. 1 shows a schematic plan view of a protective sleeve according to the invention with an integrated insertion aid,

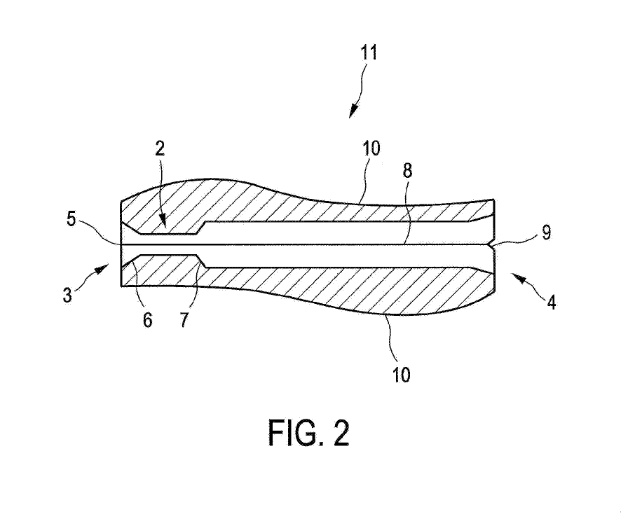

FIG. 2 shows a schematic sectional view of a further protective sleeve according to the invention with an integrated insertion aid,

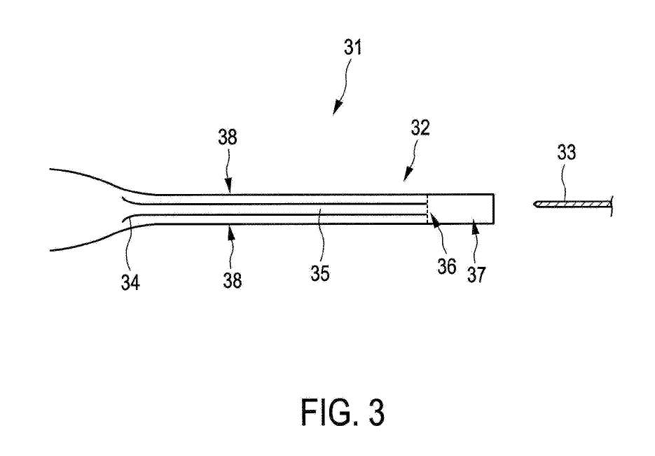

FIG. 3 shows a schematic view of a protective sleeve according to the invention in which the integrated insertion aid has a trough-like or duct-like recess (side view), and

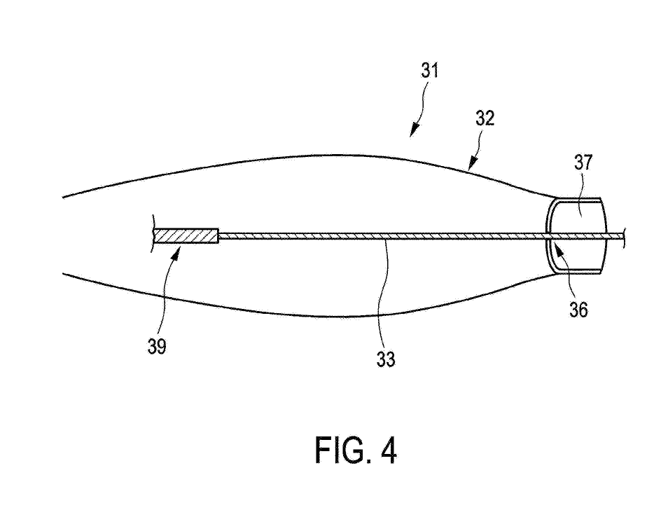

FIG. 4 shows a schematic view of the protective sleeve according to the invention from FIG. 3 (plan view).

The protective sleeve 1 according to FIG. 1, not shown in all its details, has an integrated insertion aid 2. In FIG. 1, reference sign 3 on the left is assigned to the distal end of the protective sleeve 1, and reference sign 4 on the right is assigned to the proximal end of the protective sleeve 1.

In line with the observations made in the description, a guide wire (not shown in FIG. 1) is accordingly inserted from the left-hand side of the figure (distal) into the interior of the protective sleeve 1. The actual balloon catheter (with or without stent) is situated in the interior of the protective sleeve 1; for the sake of clarity, it is not shown in FIG. 1. A balloon catheter of this kind is introduced into the interior of the protective sleeve 1 via the proximal end 4 of the protective sleeve 1. This introduction is made easier by the fact that the cross-sectional surface area (not shown in any detail in FIG. 1) widens at the proximal end 4.

The insertion aid 2 integrated in the protective sleeve is composed essentially of a tubular portion, which widens in the manner of a funnel 6 in the direction of an opening 5 provided at the distal end 3. The tubular portion with the funnel-shaped widening has a circular cross-sectional surface area in the case shown in FIG. 1. By way of the opening 5, a guide wire (not shown in FIG. 1) can be introduced into the interior of the protective sleeve 1. The funnel-shaped widening 6 ensures that it is easier to introduce/thread the guide wire into the interior of the tubular insertion aid 2.

In the direction of the proximal end 4 of the protective sleeve 1, the insertion aid 2, after the narrower tubular portion, is likewise widened in the manner of a funnel 7. Generally, the balloon catheter (with or without stent), not shown in FIG. 1, is guided from the direction of the proximal end 4 of the protective sleeve 1 to this funnel-shaped widening 7 and is secured there. This means that a guide wire, after passing the funnel-shaped widening 6 and the (narrower) tubular portion of the insertion aid 2, and after passing the funnel-shaped widening 7, slides into the interior (the inner lumen) of the balloon catheter and is guided farther through this interior of the balloon catheter. The advantageous function of the insertion aid 2, which is integrated in the protective sleeve 1, is thus illustrated clearly by FIG. 1.

Moreover, in the longitudinal direction of the protective sleeve 1, an incision 8 is provided which cuts longitudinally through the material of the protective sleeve 1 (with integrated insertion aid 2). This incision can extend through the full thickness of the material of the protective sleeve or at least through a substantial part of this material thickness. In the first case, the material of the protective sleeve is thus cut through completely, and it is (still) held together mechanically, for example by the material stress of the corresponding plastics material. In the second case, on account of the almost complete separation of the material, only a slight force is needed to bring about complete opening of the protective sleeve along the incision. A comparable solution can also be achieved by perforations in the longitudinal direction instead of the incomplete incision.

To be able to open the material of the protective sleeve more easily along the incision 8, a notch 9 is provided at the proximal end 4 of the protective sleeve 1. This allows the person using the balloon catheter according to the invention to easily take hold of the protective sleeve, and in this way the protective sleeve opened along the incision, or still to be opened, can be detached from the catheter and/or guide wire.

FIG. 2 shows a schematic sectional view of a further embodiment of a protective sleeve according to the invention with an integrated insertion aid.

For the sake of clarity, and to permit comparison with FIG. 1, the main components of the protective sleeve 11 according to FIG. 2 are designated by the same reference signs as in FIG. 1, for example the integrated insertion aid 2 with its funnel-shaped widenings 6 and 7. The distal end 3, with the opening 5 provided there, and the proximal end 4 are also provided with the same reference signs. This also applies to the incision 8 and the notch 9, with the aid of which the protective sleeve 11 can be easily opened in the manner already described.

In the protective sleeve 11 shown in FIG. 2, by comparison with the protective sleeve 1 in FIG. 1, it is simply illustrated that, for the actual advantageous function of the protective sleeve according to the invention with integrated insertion aid, the decisive aspect is the inner shape or the inner contour of the protective sleeve 1 or 11 and of the integrated insertion aid 2. The view according to FIG. 1 is tailored in particular to showing this inner contour. The outer contour of the protective sleeve 1 is not shown in detail and, in the configuration according to FIG. 1, can therefore correspond substantially to the shape of the inner contour.

By contrast, in the configuration according to FIG. 2, it is shown that the outer shape or the outer contour of the protective sleeve 11 is not of importance as regards its function according to the invention. The outer contour 10 of the protective sleeve 11 as shown in FIG. 2 can deviate in any desired manner from the inner contour shown there, which corresponds to the view in FIG. 1. It is crucial to the invention that the inner contour of the protective sleeve, together with the inner contour of the insertion aid integrated in the protective sleeve, is designed such that a guide wire (not shown) can be introduced from the distal end 3 through the insertion aid into the interior of the protective sleeve and can then be inserted farther into the balloon catheter secured in the protective sleeve.

Of the protective sleeve according to FIG. 3, it is basically only the part with the integrated insertion aid 32 that is shown, and the schematically depicted guide wire 33 is not yet inserted into the insertion aid 32.

According to FIG. 3, the insertion aid 32 of the protective sleeve 31 has, at its proximal end (shown on the left), a funnel-shaped widening 34, which is provided for receiving the catheter (not shown in FIG. 3). The opening 34 is adjoined by a tubular duct 35, which terminates in the distally arranged opening 36 of the insertion aid 32.

At the (outermost) distal end of the insertion aid 32 there is a trough-like recess 37, which is open both at the top and also distally (i.e. toward the right in FIG. 3). The guide wire can be inserted into the resulting recess 37 either from above or (as shown in FIG. 3) from the side (here the right-hand side).

The cross-sectional surface area of the recess 37 is flush with the opening 36, such that the guide wire 33, after being introduced into the recess 37, can easily slide into the opening 36 and through the duct 35 in the direction of the opening 34.

In addition, FIG. 3 shows how the insertion aid 32 of the protective sleeve 31 has flattened areas (flattenings) 38 proximally adjacent to the recess 37, both on the top and also on the bottom, which makes it easier for the user to introduce the guide wire 33 into the insertion aid 32 in the manner already described.

FIG. 4 shows a plan view of the protective sleeve 31 with insertion aid 32, specifically in the state in which the guide wire 33 is already positioned in the catheter with the aid of the insertion aid 32.

According to FIG. 4, the guide wire 33, after introduction (from the right-hand side; distal) through the opening 36 and through the duct 35 (not shown in any detail in FIG. 4), is inserted into the distal end 39 of the catheter and thus positioned in the catheter.

The features and advantages of the invention are described again below in connection with the example.

### EXAMPLE

The function of a balloon catheter according to the invention with a protective sleeve according to the invention (with integrated insertion aid) is explained again below in connection with FIGS. 1, 3 and 4.

The starting point is a balloon catheter (with or without stent), which is located in the interior of a protective sleeve 1 (with integrated insertion aid 2). According to FIG. 1, the balloon catheter is placed with its tip on the funnel-shaped widening 7 of the insertion aid 2. Advantageously, the protective sleeve 1 completely surrounds the balloon catheter (not shown in FIG. 1).

To prepare for the use of the balloon catheter, for example in PTA or PTCA, a guide wire is inserted through the opening 5, provided at the distal end 3 of the insertion aid 2, into the interior of the insertion aid 2. The opening 5 can either already be present on the insertion aid 2 or can be made with the aid of means provided there and/or with the aid of a tool (e.g. stylet). The funnel-shaped widening 6 at the distal end 3 of the insertion aid 2 and, if appropriate, the recess 37 of the insertion aid 32 facilitate the insertion of the guide wire into the interior of the insertion aid. After the guide wire has passed the insertion aid, it is (automatically) inserted/threaded into the interior (the inner lumen) of the balloon catheter, which is located inside the protective sleeve 1, and advanced through the interior of the balloon catheter. The entire insertion of the guide wire takes place in a state in which the protective sleeve 1 covers the balloon catheter either at the sensitive component parts or preferably completely. It is not necessary, nor indeed is it even possible, to touch the balloon catheter, including the coatings provided on the balloon and/or on the stent.

After the guide wire has been inserted into the interior of the balloon catheter, the protective sleeve 1 can be detached from the catheter. This preferably takes place in a state in which the balloon catheter itself is pulled out (in the proximal direction) from the interior of the protective sleeve by way of the proximal end 4 of the protective sleeve 1. In this state, the protective sleeve 1 is then still located only over the guide wire. This has, on the one hand, the advantage that the removal of the protective sleeve does not take place over possibly sensitive parts of the balloon catheter (e.g. over coatings). On the other hand, the guide wire itself has a smaller diameter than the balloon catheter, such that detachment of the protective sleeve from the guide wire is easier on account of its dimensions.

For the actual removal of the protective sleeve 1, the protective sleeve is then opened along the incision 8 and at least partially unfolded/unfurled. This is made easier by the notch 9 provided on the protective sleeve 1. The protective sleeve 1 can then be removed over the guide wire, without the balloon catheter itself being affected by this removal.

1. A balloon catheter comprising a balloon and a protective sleeve which at least partially surrounds the balloon, the balloon catheter further comprising an insertion aid for a guide wire, the insertion aid being integrated in a distal end of the protective sleeve.

2. The balloon catheter as claimed in claim 1, comprising an opening for insertion of the guide wire into an interior of the balloon catheter, the opening provided distally on the insertion aid.

3. The balloon catheter as claimed in claim 1, wherein the insertion aid has a duct-like or tubular design, and wherein the insertion aid widens like a funnel in a direction of the opening provided at the distal end.

4. The balloon catheter as claimed in claim 1, wherein a recess is configured at a distal end of the insertion aid, the recess forming a trough or an open duct.

5. The balloon catheter as claimed in claim 4, wherein the recess has a cross-sectional surface area in a shape of a segment of a circle, or has a V-shaped cross-sectional surface area.

6. The balloon catheter as claimed in claim 4, wherein a cross-sectional surface area of the recess increases toward the distal end of the insertion aid.

7. The balloon catheter as claimed in claim 4, wherein a cross-sectional surface area of the recess is flush, at its proximal end, with the opening in the insertion aid.

8. The balloon catheter as claimed in claim 4, wherein the recess is coded in order to improve discernibility of the recess to the eye.

9. The balloon catheter as claimed in claim 8, wherein the coding is provided on an inner face of the recess.

10. The balloon catheter as claimed in claim 1, wherein the protective sleeve has, proximally adjacent to the insertion aid, at least one area that is flattened in relation to a shape of a remainder of the protective sleeve.

11. The balloon catheter as claimed in claim 1, wherein the insertion aid widens proximally like a funnel, in order to receive and secure the catheter in the insertion aid.

12. The balloon catheter as claimed in claim 1, wherein the protective sleeve comprises a section for facilitating removal of the protective sleeve.

13. The balloon catheter as claimed in claim 12, wherein the section is in the form of at least one predetermined breaking point, at least one perforation and/or at least one incision in the protective sleeve.

14. The balloon catheter as claimed in claim 12, wherein the section comprises at least one handle for removal of the protective sleeve.

15. A protective sleeve for a balloon catheter comprising a balloon, the protective sleeve configured to at least partially surround the balloon, wherein an insertion aid for a guide wire is integrated in the protective sleeve.

16. (canceled)

17. A method for inserting a guide wire into an interior of a balloon catheter, the method comprising the steps of:
providing a balloon catheter, the balloon catheter having a protective sleeve which at least partially surrounds the balloon, wherein an insertion aid is integrated in a distal end of the protective sleeve; and
inserting the guide wire distally into the protective sleeve.

18. The method as claimed in claim 17, wherein a recess is configured at a distal end of the insertion aid, or is integrally formed on the insertion aid, the recess forming a trough or an open duct.

19. The balloon catheter of claim 1 further comprising a stent, wherein the protective sleeve at least partially surrounds the stent.

20. The protective sleeve of claim 15, wherein the protective sleeve is configured to at least partially surround a stent on the balloon catheter.
